(19) United States
(12) Patent Application Publication
(10) Pub. No.: US 2020/0206732 A1
(43) Pub. Date: Jul. 2, 2020

(54) METHODS AND APPARATUS FOR COLLECTING AND TESTING SOLID OR SEMI-SOLID MATERIALS

(71) Applicant: Evolve BioSystems, Inc., Davis, CA (US)

(72) Inventor: Bethany Henrick, Davis, CA (US)

(21) Appl. No.: 16/237,029

(22) Filed: Dec. 31, 2018

(51) Int. Cl. B01L 3/00; A61B 10/00

(52) U.S. Cl. CPC B01L 3/50825; A61B 10/0096; B01L 3/565; B01L 2300/042; B01L 2300/0832; B01L 2300/041; B01L 2300/0858; B01L 3/523

(57) ABSTRACT

A collection and test apparatus is provided that comprises a collection wand, a tubular test housing and an annular seal member. The collection wand has a cylindrical stem and a collection tip. The collection tip has a cylindrical sample capture portion having one or more specimen capture grooves each having a groove volume and collectively providing a specimen capture volume. The annular seal member has a seal aperture sized to allow passage of the cylindrical sample capture portion of the collection tip. The seal member is fixedly positioned within the tubular housing transverse to its longitudinal axis and divides the housing into a proximal housing space and a distal housing space comprising a test chamber sized to receive an amount of reagent liquid. The collection wand is positionable in a test configuration where the collection tip is disposed within the test chamber and the stem extends through the aperture seal.

### BACKGROUND OF THE INVENTION

The present invention relates generally to devices for collecting and analyzing solid material samples. More specifically, the invention relates to a devices configured to provide precise amounts of solid or semi-solid material for mixture with one or more liquid reagents for analysis of the material.

Devices for collecting and testing biological specimens are well-known. Such devices typically involve the use of a swab or similar collection device to capture cells from a biological specimen. This is generally accomplished by bringing the tip of the device into contact with the specimen so that a quantity of biological material adheres to it. The tip is then inserted into a tube or other chamber where it can be brought into contact with a chemical reagent selected so as to indicate the presence of a particular compound or agent within the specimen.

Also well-known are self-contained test devices that include both a swab-tipped collection wand and a receiving container having a test chamber and a separate reagent-filled reservoir. These devices are configured so that the reagent can be selectively added to the test chamber after the wand tip (with a collected biological material) has been positioned therein. Examples of devices of this type can be found in U.S. Pat. Nos. 4,978,504; 5,266,266; 5,869,003; and 5,879,635.

The above-described devices are generally concerned with providing enough of a sample to achieve a reaction when contacted by a reagent. They are not intended and, indeed, are not capable of providing a precise, known quantity of specimen material for reaction with a known quantity of reagent.

### SUMMARY OF THE INVENTION

An illustrative aspect of the invention provides a collection and test apparatus comprising a collection wand, a tubular test housing and an annular seal member. The collection wand comprises an elongate, cylindrical stem with a stem diameter and proximal and distal stem ends, and a collection tip extending distally from the distal stem end. The collection tip comprises a cylindrical sample capture portion having one or more specimen capture grooves formed therein. The sample capture portion has a capture portion diameter that is greater than or equal to the stem diameter. Each of the one or more capture grooves has a groove volume and collectively the one or more capture grooves provide a specimen capture volume. The tubular test housing has a housing interior, an open proximal end and a closed distal end, and a longitudinal housing axis extending through the proximal and distal ends. The annular seal member has a seal aperture sized to allow passage of the cylindrical sample capture portion of the collection tip there-through. The seal member is fixedly positioned within the tubular housing transverse to the longitudinal housing axis and divides the housing interior into a proximal housing space and a distal housing space comprising a test chamber sized to receive at least a predetermined amount of reagent liquid. The collection wand is positionable in a test configuration in which the collection tip is disposed within the test chamber and the stem extends through the aperture seal and distally outward through the open proximal end of the test housing. In some illustrative embodiments, at least a portion of the stem is a tube having a stem fluid passage formed there-through. The stem fluid passage has a proximal stem passage opening through the proximal stem end and a distal passage opening formed through a wall of the tube at a location that is distal to the seal member when the collection wand is positioned in the test configuration. In further illustrative embodiments, the collection wand may comprise a handle portion attached at the distal end of the stem. The handle portion comprises a resiliently deformable squeeze bulb defining a reagent reservoir. The handle further comprises a removable passage closure at the proximal stem passage opening, the passage closure preventing fluid flow through the proximal stem passage opening. Removal of the passage closure places the test chamber in fluid communication with the reagent reservoir via the stem fluid passage.

Another illustrative aspect of the invention provides a method of testing a solid or semi-solid target material using a collection and test apparatus having a collection wand and a tubular test housing with an annular seal disposed therein. The collection wand has a collection tip with a cylindrical sample capture portion with one or more specimen capture grooves collectively providing a specimen capture volume. The tubular test housing is configured for receiving the collection tip through a proximal opening of the test housing and through a seal aperture of the annular seal member into a distal test chamber. The method comprises capturing target material by contacting the target material with the collection tip of the collection wand so as to at least completely fill each of the one or more specimen capture grooves. The method further comprises placing a predetermined amount of reagent liquid in the distal test chamber. The method still further comprises inserting the collection tip through the proximal opening of the test housing and passing the collection tip through the seal aperture, thereby removing captured target material adhered to the sample capture portion outside the specimen capture grooves. The method also comprises positioning the collection wand in a test configuration in which the collection tip and remaining captured target material are immersed in the reagent liquid in the distal test chamber and dispersing the remaining captured target material in the reagent liquid.

These and other objects, features, and advantages of the present invention will appear more fully from the following description of the exemplary embodiments, taken in conjunction with the accompanying drawings.

### BRIEF DESCRIPTION OF THE DRAWINGS

FIG. 1 is a side view of a collection and test apparatus according to an exemplary embodiment of the invention in which the collection wand is separated from the test housing;

FIG. 2A is a side view of the collection and test apparatus of FIG. 1 in which the collection wand is in its test configuration;

FIG. 2B is a section view of the collection and test apparatus of FIG. 1 in which the collection wand is in its test configuration;

FIG. 3A is a side view of a collection tip of a collection and test apparatus according to an embodiment of the invention;

FIG. 3B is a section view of a collection tip of a collection and test apparatus according to an embodiment of the invention;

FIG. 4A is a side view of a collection tip of a collection and test apparatus according to an embodiment of the invention;

FIG. 4B is a section view of a collection tip of a collection and test apparatus according to an embodiment of the invention;

FIG. 5A is an end view of a seal of a collection and test apparatus according to an embodiment of the invention;

FIG. 5B is a section view of a seal of a collection and test apparatus according to an embodiment of the invention;

FIG. 5C is a section view of a seal of a collection and test apparatus according to an embodiment of the invention;

FIGS. 6A-6D are section views illustrating the passage of a specimen laden collection tip through a seal according to an embodiment of the invention;

FIG. 7 is a block diagram of a collection and test method according to embodiments of the invention;

FIG. 8 is a side view of a collection and test apparatus according to an embodiment of the invention with reagent liquid in the test chamber and the collection wand in its test configuration;

FIG. 9A is a side view of a collection and test apparatus according to an embodiment of the invention;

FIG. 9B is a section view of the collection and test apparatus of FIG. 9A;

FIG. 10 is a section view of a portion of the collection and test apparatus of FIG. 9A;

FIG. 11 is a section view of a portion of a collection and test apparatus according to an embodiment of the invention;

FIG. 12 is a block diagram of a collection and test method according to embodiments of the invention;

FIG. 13A is a side view of a collection and test apparatus according to an embodiment of the invention;

FIG. 13B is a section view of the collection and test apparatus of FIG. 13A;

FIG. 14 is a section view of a portion of the collection and test apparatus of FIG. 13A;

FIG. 15 is a block diagram of a collection and test method according to embodiments of the invention;

FIG. 16 is a section view of a portion of a collection and test apparatus after certain actions of a method according to an embodiment of the invention have been carried out;

FIG. 17 is a section view of a portion of a collection and test apparatus after certain actions of a method according to an embodiment of the invention have been carried out; and

FIG. 18 is a section view of a collection and test apparatus after certain actions of a method according to an embodiment of the invention have been carried out.

### DETAILED DESCRIPTION OF THE INVENTION

Hereinafter, aspects of the invention in accordance with various exemplary embodiments will be described. The present invention provides methods and apparatus for collecting and testing solid or semi-solid material (e.g., stool) where the maximum amount of solid or semi-solid material is controllable. Various embodiments of the invention make use of a collection wand having a solid material collecting tip that is insertable through a close or interference fitting aperture into a reaction chamber. The wand tip and the aperture are configured so that when the tip is passed through the aperture, solid material in excess of a desired amount is removed from the tip and prevented from entering the reaction chamber.

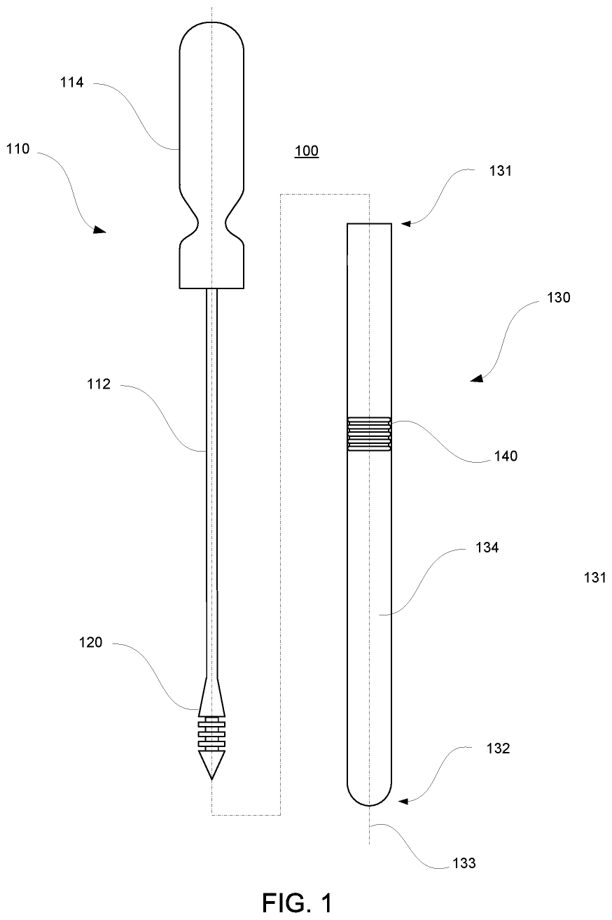

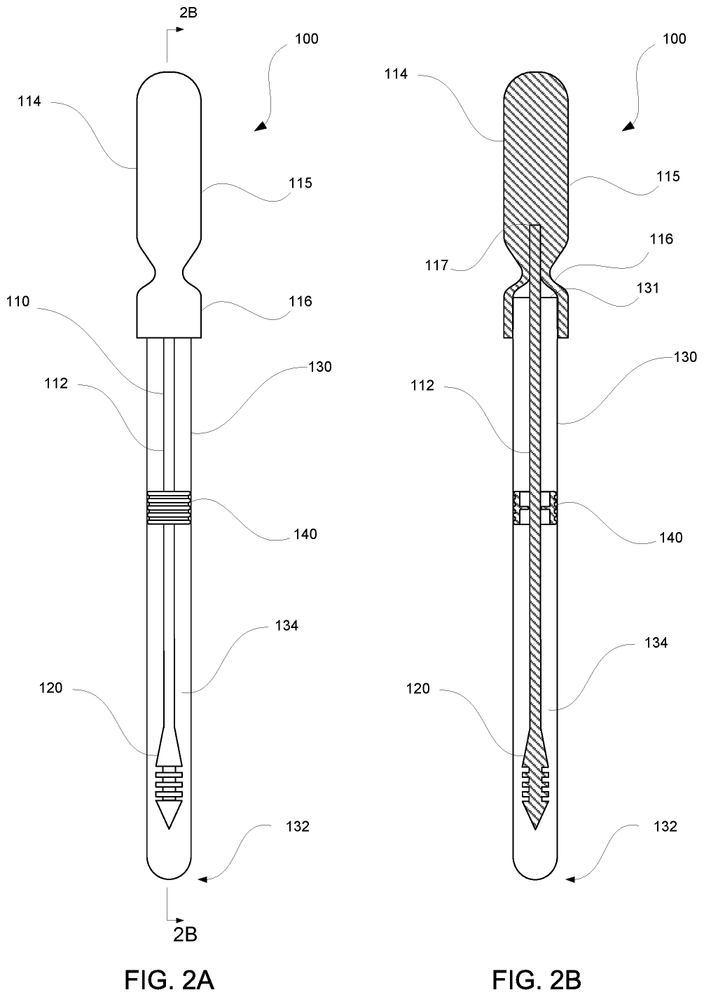

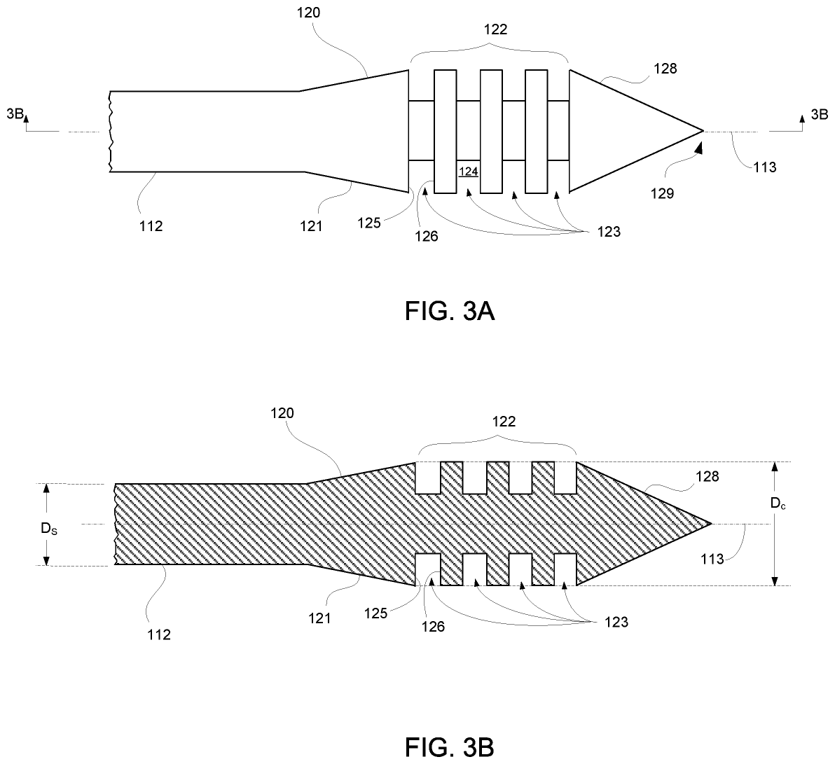

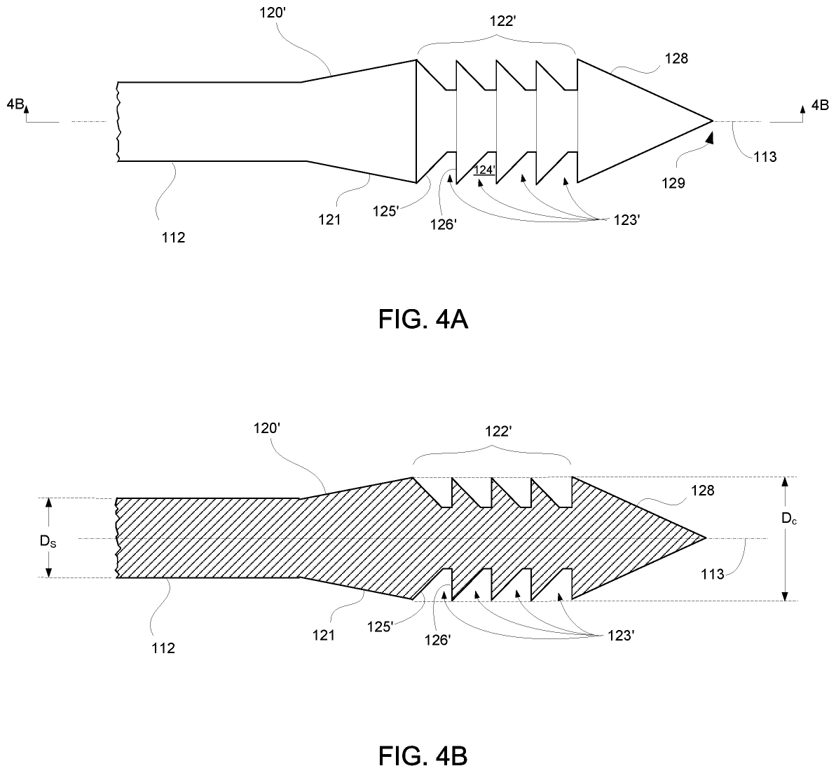

Embodiments of the invention will now be discussed in more detail. With reference to FIGS. 1-6, a specimen collection and test apparatus 100 according to an embodiment of the invention includes a collection wand 110 and a tubular test housing 130 with an annular seal 140 disposed therein. The collection wand 110 has a collection tip 120 configured for capturing solid or semi-solid material. FIGS. 2 and 3 illustrate the test apparatus in a testing configuration in which the tip portion of the wand has been inserted into the tubular test housing 130 and through the annular seal 140 into a test chamber 134.

The tubular test housing 130 is axisymmetric about a tube axis 133 extending from and through an open proximal end 131 and to and through a closed distal end 132. The tubular housing 130 may be formed from any suitable material such as molded plastic or glass. Some or all of the tubular housing 130 may be transparent to allow the observation of material placed therein.

The collection wand 110 has a cylindrical stem 112 attached at its proximal end to a handle 114. A collection tip 120 extends from the stem 112 at its distal end. The collection tip 120 is axisymmetric with respect to the longitudinal axis 113 of the stem 112. The collection tip 120 has a cylindrical section 122 with a larger diameter than the stem 112, a frusto-conical transition section 121 that connects the cylindrical section 122 to the stem 112, and a conical end section 128 terminating at a distal end 129. It will be understood that in some embodiments of the invention, the stem 112 could have the same diameter as the cylindrical section 122. In such embodiments, the transition section 121 may not be present.

The cylindrical section 122 of the collection tip 120 has a plurality of circumferential grooves 123 formed therein that collectively define a collection volume 124. The number, size and geometric configuration of the grooves 123 can be selected in order to provide a desired volume for collection of specimen material. In the embodiment illustrated in FIGS. 2A and 2B, the grooves 123 are equal in size and have a substantially rectangular cross-section in which the proximal and distal walls 125, 126 of each groove are orthogonal to the stem axis 113. It will be understood, however, that the grooves can be formed with varying sizes and shapes. FIGS. 3A and 3B illustrate an alternative collection tip 120′ having a cylindrical section 122′ with a plurality of grooves 123′ formed therein. In this embodiment, the proximal wall 125′ of each groove 123′ is angled in a distal direction from the orthogonal while the distal wall 126′ remains orthogonal to the tip axis 113. The effect is to present a “fishbone” appearance to the collection tip 120′. In variations of this pattern, either or both of the proximal and distal groove walls 125′, 126′ may be angled and the degree of angle may be changed.

In the illustrated embodiments, the grooves 123, 123′ all have substantially the same geometry. In other embodiments, however, the geometry and volume of the grooves 123, 123′ may be different. In all cases, however, the grooves 123, 123′ may be configured so that they provide a desired cumulative collection volume 124 that is bounded by the diameter D_{C} of the cylindrical section 122.

The collection tip 120 may be formed from any material that is sufficiently rigid to maintain a consistent geometry and collection volume during use. This may include but is not limited tovarious plastic materials. In particular embodiments, the collection tip 120 is formed from one or more polyethylenes. In some embodiments, the collection tip 120 may be integrally formed with the wand stem 112. Alternatively, the collection tip 120 may be separately formed and bonded to or otherwise attached to the distal end of the stem 112.

The handle 114 has a grip portion 115 fixedly attached at the proximal end 117 of the wand stem and a cap portion 116 configured to fit over and seal the open end 131 of the tubular housing 130.

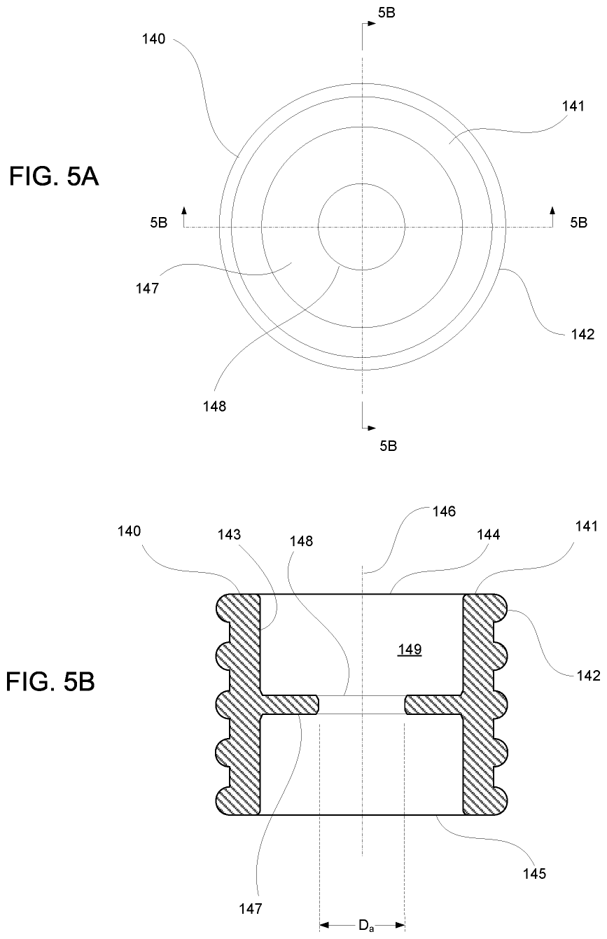

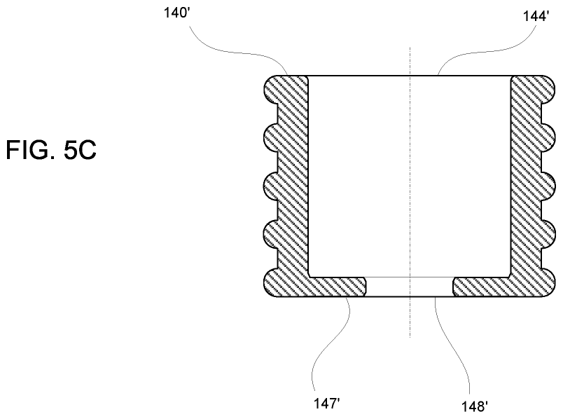

The seal 140 has a generally cylindrical body 141 sized to fit within and engage the interior surface of the tubular test housing 130. The outer surface of the seal body 141 may be cylindrical with a diameter the same or similar to the inside surface of the tubular housing 130. Alternatively, as shown in the illustrated embodiment and best seen in FIGS. 5A and 5B, the outer surface may be formed as a smaller diameter cylinder with circumferential ridges 142 extending outwardly therefrom to contact the inner surface of the tubular housing. The seal body 141 has a cylindrical seal passage 149 formed there-through, the seal passage 149 being bounded by an inner seal surface 143. The cylindrical seal passage 142 extends from a proximal seal entrance plane 144 to a distal seal exit plane 145 along a seal centerline 146.

The seal body 141 may be disposed within the tubular test housing 130 at a position intermediate the open end 131 of the housing 130 and the test chamber 134. The seal body 141 may be bonded in place or held in place through an interference fit with respect to the tubular housing 130. In some embodiments, additional retaining rings may be positioned distal and/or proximal to the seal body to hold it in place.

The seal 140 further includes an annular seal member 147 positioned within and transversely across the seal passage 149. The annular seal member 147 may be in the form of an annular disc as shown in the illustrated embodiment or may be in the form of a tapered or frusto-conical annular member. The seal member 147 has a circular seal aperture 148 formed there-through. As shown in FIG. 5B, the annular seal member 147 may be positioned intermediate the entrance plane 144 and the exit plane 145. Alternatively, the annular seal member 147 may be positioned at or adjacent the entrance plane 144 or the exit plane 145. FIG. 5C illustrates an alternative seal 140′ having a proximal entrance plane 144′, a distal exit plane 145′ and an annular seal member 147′ positioned adjacent the distal exit plane 145′. In this configuration, the distal end of the seal aperture 148′ is positioned at the distal exit plane 145′.

The seal aperture 148 has a diameter Dₐ that is selected so as to allow passage of the collection tip 120 there-through with little or no clearance between the circumferential edge of the aperture 148 and the outer surface of the cylindrical portion 122 of the collection tip 120.

In some embodiments, the seal aperture diameter Dₐ may be selected so that it is just equal to or slightly greater than the maximum diameter D_{c} of the collection tip 120 (i.e., the diameter of the cylindrical portion 122). In such embodiments, when the collection tip 120 is passed through the seal aperture 148, the close (or zero) clearance between the cylindrical portion 122 of the collection tip 120 and the perimeter of the seal aperture 148 assures that material adhered to the cylindrical portion 122 that is outside the cylindrical boundary of the cylindrical portion 122 is scraped off the collection tip 120 and retained with in the seal body. Such embodiments have the disadvantage, however, that if the stem diameter Dₛ is smaller than the maximum diameter D_{c} of the collection tip 120, there will be a gap between the stem 112 and the perimeter of the seal aperture 148 after the cylindrical portion 122 has passed through. This could allow scraped off material to fall or otherwise pass through the aperture 148.

To avoid this problem, the seal member 147 may be formed from a pliable material that is resiliently deformable and allows the seal aperture 148 to expand to accommodate the passage of the collection tip 120 through it. In such embodiments, the initial seal aperture diameter Dₐ is initially smaller than the maximum diameter D_{c} of the collection tip 120, but expands to the maximum diameter D_{c} when the collection tip is pushed through it with moderate force. After passage of the cylindrical portion of the collection tip 120, the resilience of the material returns the seal member 147 and the seal aperture 148 to their original configuration.

Any suitably pliable and resilient material may be used to form the seal member 147 including, but not limited tosilicone and polyethylene. The seal member 147 may be integrally formed with the seal body 141 or may be separately formed from the same or different material and attached to the seal body 141 (e.g., by bonding). In some embodiments, the seal member 147 and the seal body 141 may be collectively formed from multiple components.

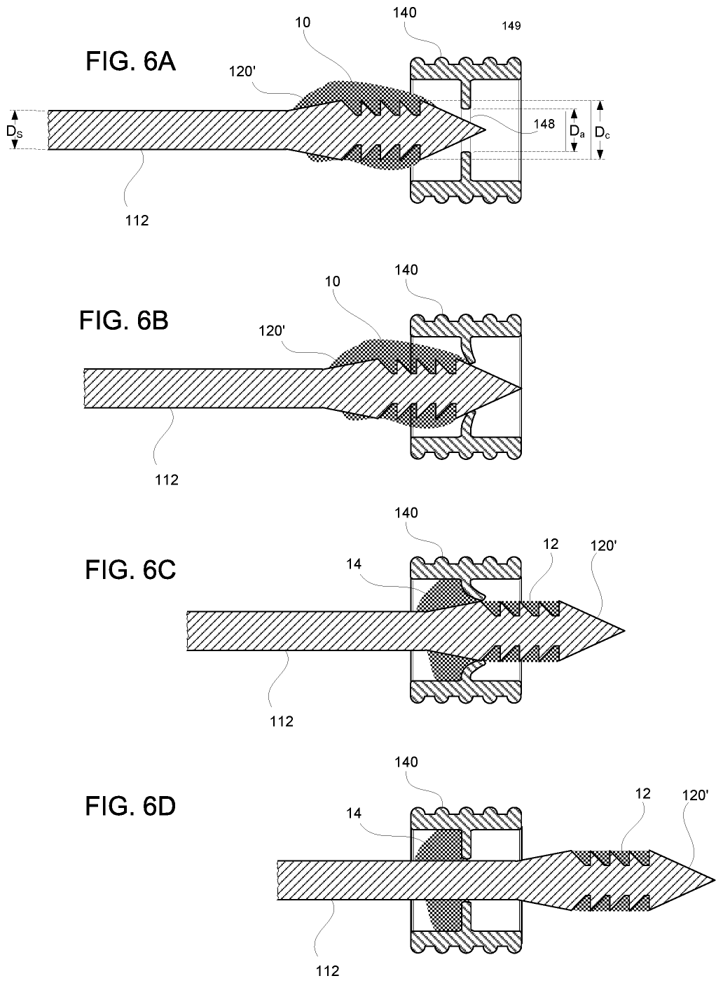

FIGS. 6A-6D illustrate the sequence as a collection tip 120′ laden with specimen material 10 is passed through a seal 140 having an aperture 148 with an undeformed aperture diameter Dₐ (e.g., 0.14 in.) that is smaller than the maximum diameter D_{c} (e.g., 0.20 in.) of the collection tip 120′. As is shown in FIGS. 6B and 6C, once the seal member 147 is engaged by the collection tip 120′, further distal translation of the collection tip 120′ causes the seal member 147 to deform and the aperture 148 to expand so as to just equal or exceed the maximum tip diameter D_{c}. As the collection tip 120′ passes through the seal aperture 148, only the desired specimen material 12 retained within the grooves of the collection tip 120′ pass through with it. Excess specimen material 12 is scraped off and retained within the seal passage 149. As shown in FIG. 6D, after passage of the collection tip 120′ through the aperture 148, the aperture 148 returns to its original diameter.

In the embodiment illustrated in FIGS. 6A-6D, the aperture diameter Dₐ is slightly larger than the stem diameter Dₛ. In other embodiments, the aperture diameter Dₐ may be the same or slightly smaller than the stem diameter Dₛ so that there is no gap between the wand stem 112 and the perimeter of the aperture 148 after the collection tip 120 has passed through. In such embodiments, the seal member 147 may literally act to seal off the portion of the test housing 130 proximal to the seal member 147 from the portion of the housing 130 (including the test chamber 134) that is distal to the seal member 147.

It will be understood that the diameters of the wand stem 110 and the cylindrical portion 122 of the collection tip 120, the material and geometry of the seal member 147, and the seal aperture diameter Dₐ can be selected in combination to provide the penetration resistance, material retention/passage prevention, and sealing characteristics desired for a given application.

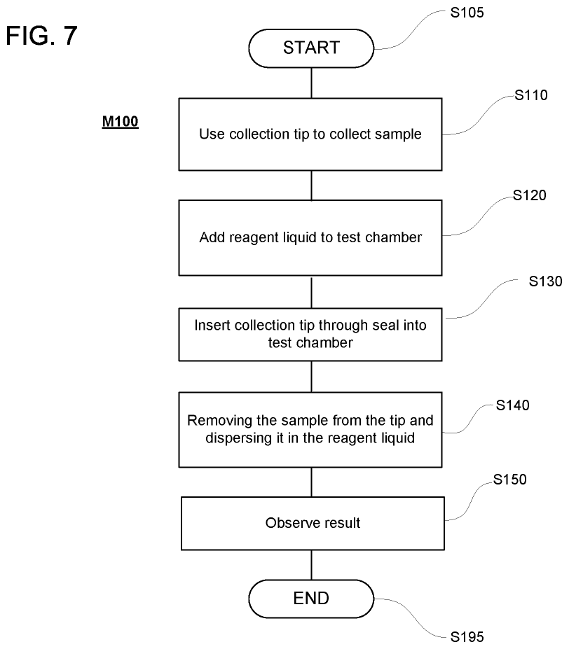

The actions of a method M100 of using the collection and test apparatus 100 according to an embodiment of the invention is shown in FIG. 7. The specimen collection and test apparatus 100 may be provided to a user as two separate components as shown in FIG. 1 or combined in the test configuration shown in FIGS. 2A and 2B. If provided in the test configuration, a user must first separate and withdraw the wand 110 from the test housing 130. The method M100 begins at S105 and at S110 the user uses the collection tip 120 of the wand 110 to collect a sample of solid or semi-solid material. This is generally done by rolling the collection tip 120 through the target material (e.g., stool) to adhere the material to the collection tip 120. The action may be repeated until all of the one or more grooves 123 of the tip 120 are filled with the target material. The user need not be concerned regarding excess material adhering to the tip and, indeed, may be instructed that too much material is acceptable while too little may invalidate the test. At S120, a reagent liquid is added to the test chamber 134 of the tubular test housing 130. This can be accomplished by simply pouring or injecting the liquid into the open proximal end of the test housing 130, through the seal 140 and into the test chamber 134. The amount of liquid added is preferably a predetermined amount calculated to provide a desired ratio of reagent liquid to the amount of sample material in the grooves 123 of the collection tip 120. In some embodiments, a line indicator may be provided on the test housing 130 to show the level to which the user should fill the test chamber 134 with the reagent fluid. The reagent fluid itself may comprise any one or more reagents desired to provide a particular reaction with the target sample material.

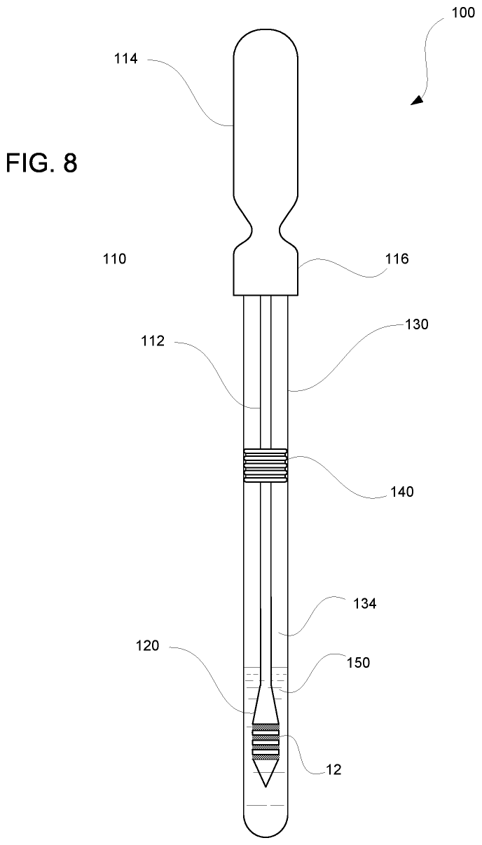

At S 130, the user inserts the collection tip 120 through the open end of the test housing 130 and presses it through the seal aperture 148 of the seal 140, thereby removing excess sample material from the collection tip 120. The user continues to move the tip distally into the test chamber 134 where the tip 120 and the final test specimen it is fully immersed in the reagent fluid 150 as shown in FIG. 8. In this configuration, the cap portion 116 of the wand handle 114 may serve to seal off the proximal end 131 of the test housing 130. At S140, the test housing 130 and wand 110 may be agitated to assure that all of the specimen material 12 is removed from the collection tip 120 and mixed with the reagent liquid 150. At S150, the user observes or otherwise determines a test result based on the reaction of the specimen material 12 with the reagent liquid 150. This could, for example, be a change in the color of the liquid 150, which would be observable through the wall of the tubular housing 130. Such a color change could be compared to a color key provided with the test apparatus 100. In particular embodiments, this color key could be applied to the housing 130 itself. The method ends at S195.

The embodiments discussed above require that the reagent liquid be provided separately from the test apparatus and manually added to the test chamber prior to insertion of the material-laden collection tip. The embodiments that follow provide test apparatus in which one or more reagent fluids may be stored within the apparatus and added to the test chamber after insertion of a material-laden collection tip.

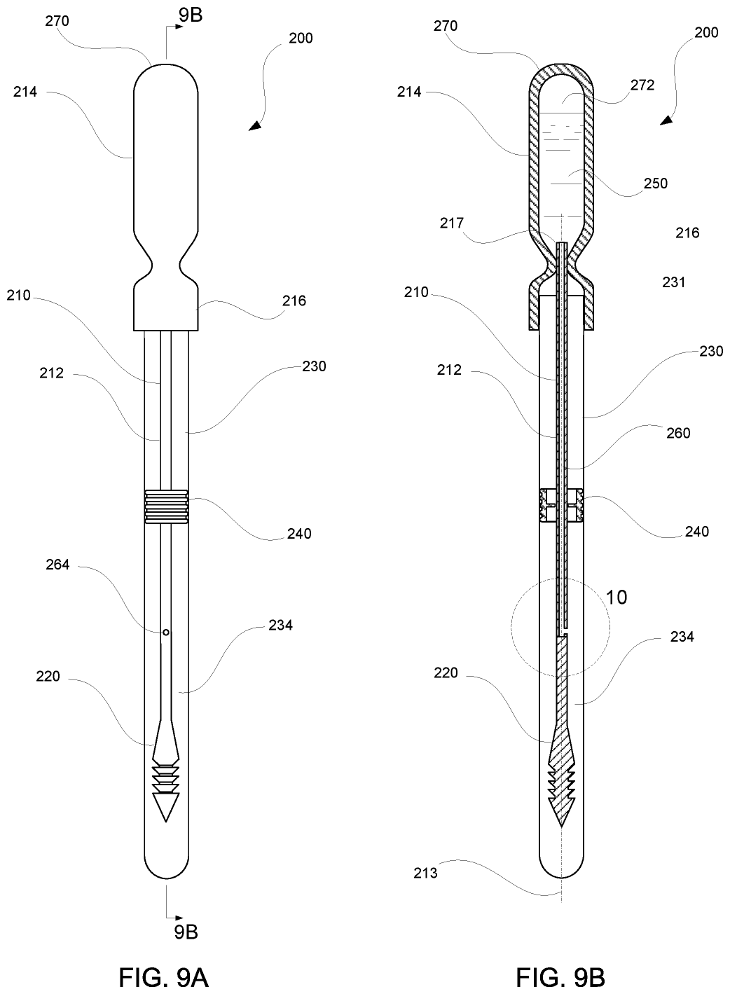

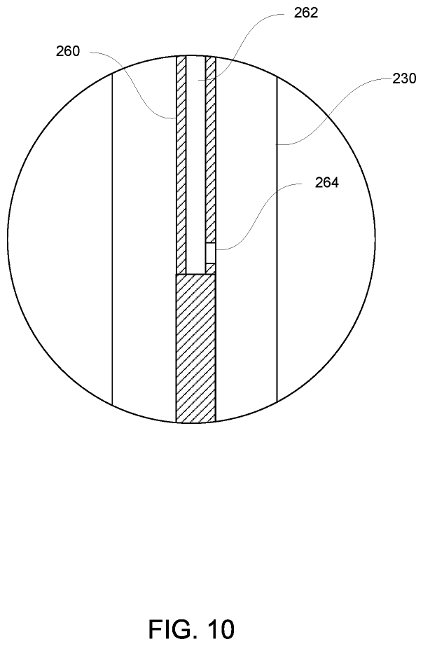

With reference to FIGS. 9A and 9B, a specimen collection and test apparatus 200 according to an embodiment of the invention includes a collection wand 210 and a tubular test housing 230 with an annular seal 240 disposed therein. The tubular test housing 230 and the annular seal 240 are substantially similar to the test housing 130 and seal 140 of the apparatus shown in FIGS. 1-6. The collection wand 210 has a collection tip 220 that is substantially similar to the collection tip 120′ shown in FIGS. 4A and 4B. It will be understood, however, that any collection tip usable in the collection and test apparatus 100 of FIGS. 1-6 is also usable in the apparatus 200 of FIGS. 9a and 9B.

As in the previous embodiments, the annular seal 240 and the collection tip 220 may be jointly configured so that when the collection tip 220 is inserted through the annular seal 240, excess specimen material outside the grooves of the collection tip 220 is prevented from passing through the seal 240. In some embodiments, the seal 240 may be configured to substantially seal off the test chamber 234 from the interior of the housing 230 proximal to the seal 240.

The collection wand 210 comprises a cylindrical stem 212 attached at its proximal end to a handle 214 and at its distal end to the collection tip 220. In this embodiment, some or all of the stem 212 is formed as a tube 260 having a fluid passage 262 formed therethrough. The tube 260 is open at its proximal end 217 and has a lateral opening 264 at a location that is distal to the seal 240 when the collection wand 210 is in the test configuration shown in FIGS. 9A and 9B.

In this embodiment of the invention, the handle 214 of the collection wand 210 comprises a resiliently deformable squeeze bulb 270 the interior of which acts as a reservoir 272 for a reagent liquid 250. The squeeze bulb 270 has a distal opening that surrounds and is attached to the fluid passage tube 260 at or adjacent its open proximal end 217. The handle 214 has a cap portion 216 configured to fit over and seal the open end 131 of the tubular housing 130 when the collection tip 220 is fully inserted and the wand 210 is in the test configuration.

When in the test configuration, it can be seen that the fluid passage 262 provides fluid communication between the reservoir 272 and the test chamber 234. This provides a path for the reagent liquid 250 past the seal 240 when the stem 210 is disposed there-through. This allows a user to position the material-laden collection tip 220 in the test chamber 234 and then introduce the reagent liquid 250 by compressing the squeeze bulb 270 to force the liquid out of the reservoir 272, through the fluid passage 262 and out through the opening 264 into the test chamber 234.

Methods of using the collection and test apparatus 200 may be substantially similar to those previously discussed in relation to the apparatus 100 of FIGS. 1-6. The primary difference is that the action of adding reagent liquid to the test chamber can be carried out after the collection tip has been inserted through the seal into the test chamber, and the action can be carried out by forcing the reagent liquid from the reservoir 272 into the test chamber 234 as discussed above. These methods may also include placing a predetermined amount of the reagent fluid into the reservoir 272. In some embodiments, this could be accomplished by drawing fluid through the passage 262 into the reservoir 272 when the wand 210 is removed from the test housing 230.

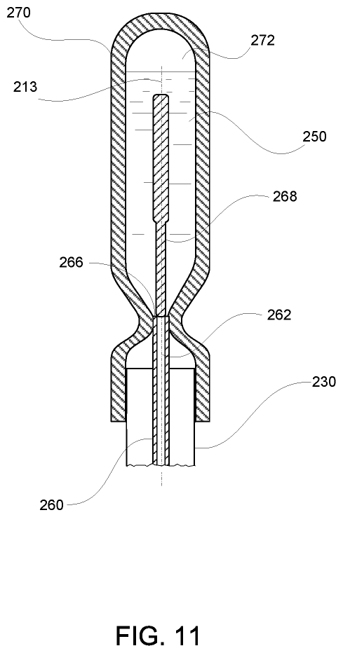

To provide for longer-term storage of the reagent liquid 250 within the reservoir 272, the tubular portion 260 of the stem 210 may be provided with a frangible closure 266 as shown in FIG. 11. The closure 266 serves to close off the proximal end 217 of the stem tube 260, thereby preventing reagent liquid 250 from exiting the reservoir 272. A break-off nib 268 may be provided that extends proximally away from the frangible closure 266 into the reservoir 272. The frangible closure 266, break-off nib 268 and squeeze bulb 270 are configured so that when the squeeze bulb 270 and nib 268 are bent through at least a predetermined angle (typically 25 to 40 degrees) from the longitudinal axis 213 of the stem 210, the frangible closure 266 breaks away to open the passage 262 to the reagent liquid 250 in the reservoir 272. This allows the provider of the apparatus 200 to pre-store a predetermined amount of reagent liquid 250 in the reservoir.

It will be understood that other selectively openable closure mechanisms may be provided for the proximal end of the stem tube 260. Any such closure providing for the long term storage of a reagent liquid 250 and selectively removable or openable to allow passage of the reagent liquid 250 into and through the stem tube 260 after collection and insertion of the stem into the test housing 230 may be used.

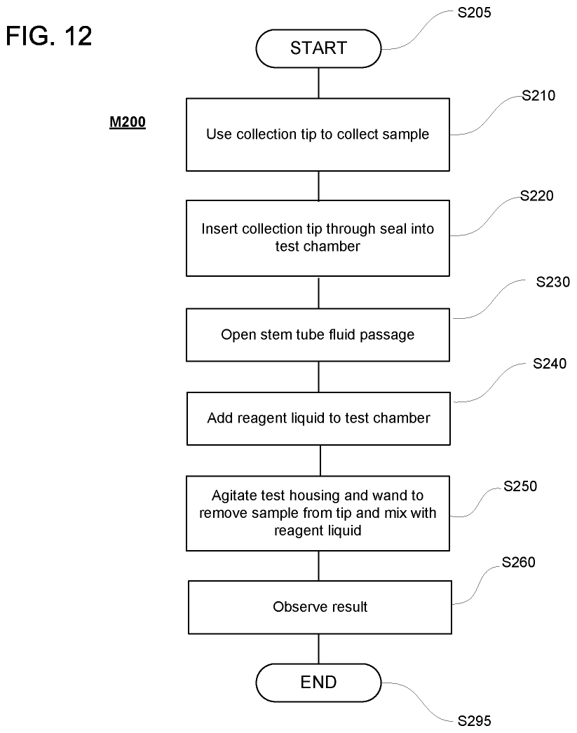

With reference to FIG. 12, a method M200 of using the collection and test apparatus 200 provided with the closure mechanism of FIG. 11 begins at S205. At S210, the user uses the collection tip 220 of the wand 210 to collect a sample of solid or semi-solid material. As before, this may typically be accomplished by rolling the collection tip 220 through the target material (e.g., stool) to adhere the material to the collection tip 220. The action may be repeated until all of the grooves of the tip 220 are filled with the target material. At S220, the user inserts the collection tip 220 through the open end of the test housing 230 and presses it through the aperture of the seal 240, thereby removing excess sample material from the collection tip 220. The user continues to move the tip distally into the test chamber 234 and into the test configuration. At S230, the user opens the stem tube fluid passage by bending the squeeze bulb 270 and nib 268 at least the predetermined fracture angle to break the frangible closure 266. At S240, the user adds reagent to the test chamber 234 by compressing the squeeze bulb 270, thereby forcing reagent liquid 250 out of the reservoir 272, into and through the passage 262 and out through the opening 264. Upon completion of this action, the sample-laden tip 220 is fully immersed in the reagent fluid 250. At S250, the test housing 230 and wand 210 may be agitated to assure that all of the specimen material is removed from the collection tip 220 and mixed with the reagent liquid 250. At S260, the user observes or otherwise determines a test result based on the reaction of the specimen material 12 with the reagent liquid 250. This could, for example, be a change in the color of the liquid 250, which would be observable through the wall of the tubular housing 230. Such a color change could be compared to a color key provided with the test apparatus 200. In particular embodiments, this color key could be applied to the housing 230 itself. The method ends at S295.

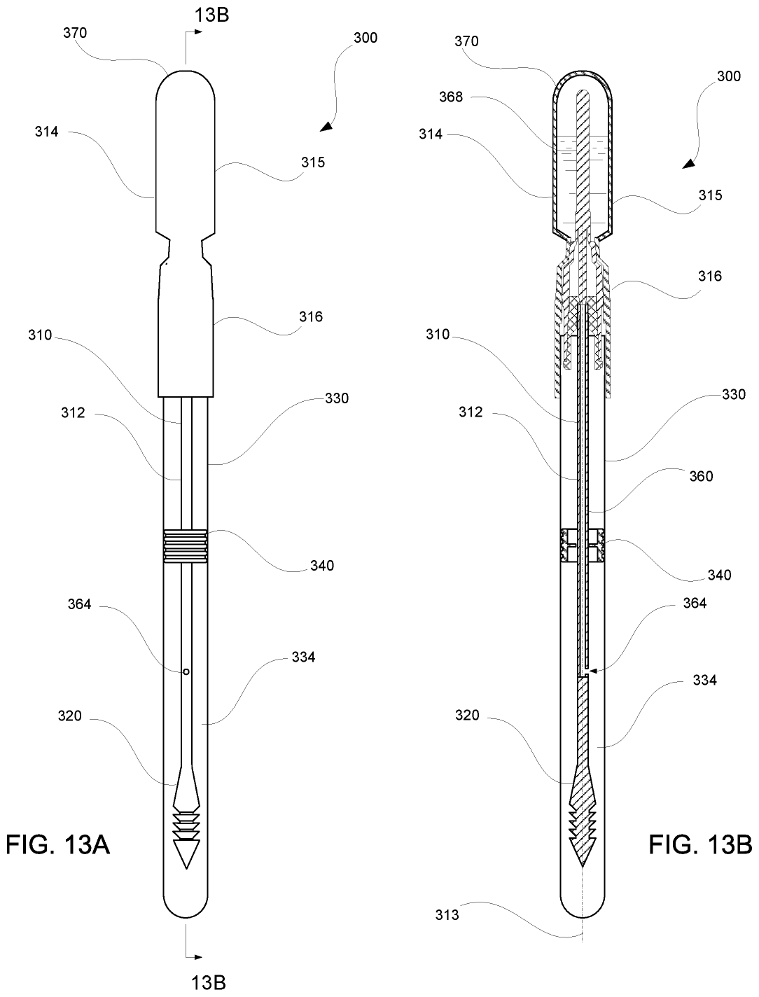

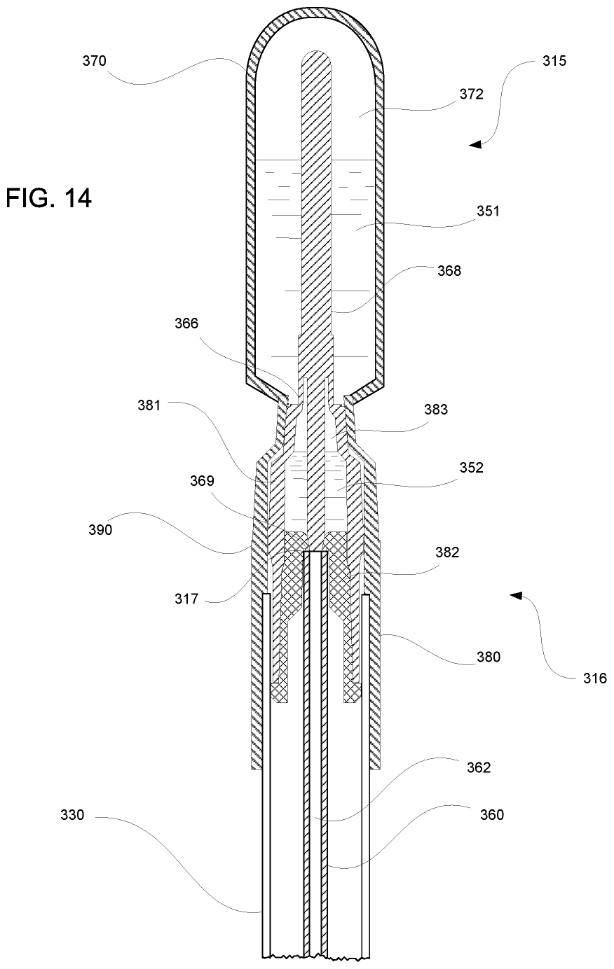

The foregoing embodiment is usable for any reagent or combination of reagents that can be combined for relatively long term storage. In some applications, reagents cannot be blended and stored in advance. Such combinations must be made immediately prior to introducing them to a sample material. FIGS. 13A, 13B and 14 illustrate a collection and test apparatus 300 according to an embodiment of the invention that provides for separate storage of two reagent liquids that can be combined after a sample is collected and positioned for testing. The collection and test apparatus 300 includes a collection wand 310 and a tubular test housing 330 with an annular seal 340 disposed therein. The tubular test housing 330 and the annular seal 340 are substantially similar to the test housing 130 and seal 140 of the apparatus shown in FIGS. 1-6. The collection wand 310 has a collection tip 320 that is substantially similar to the collection tip 120′ shown in FIGS. 4A and 4B. It will be understood, however, that any collection tip usable in the collection and test apparatus 100 of FIGS. 1-6 is also usable in the apparatus 300 of FIGS. 13A, 13B and 14.

As in the previous embodiments, the annular seal 340 and the collection tip 320 may be jointly configured so that when the collection tip 320 is inserted through the annular seal 340, excess specimen material outside the grooves of the collection tip 320 is prevented from passing through the seal 340. In some embodiments, the seal 340 may be configured to substantially seal off the test chamber 334 from the interior of the housing 330 proximal to the seal 340.

The collection wand 310 comprises a cylindrical stem 312 attached at its proximal end to a handle 314 and at its distal end to the collection tip 320. As in the apparatus 200 of the previous embodiment, some or all of the stem 312 is formed as a tube 360 having a fluid passage formed there-through. The tube 360 has a lateral opening 364 at a location that is distal to the seal 340 when the collection wand 310 is in the test configuration shown in FIGS. 13A and 13B.

With reference, in particular, to FIG. 14, the handle 314 of the collection wand 310 has a proximal portion 315 and a distal portion 316. The proximal portion 315 includes a resiliently deformable deformable squeeze bulb 370, the interior of which acts as a first fluid reservoir 372 for retaining a first reagent liquid 351. In some embodiments, the squeeze bulb 370 may be formed from a transparent material to allow observation of the fluid or fluids in the first reservoir 372. The distal portion 316 includes a cap housing 390 having an outer cap portion 380, a distal portion of which is configured to surround and engage the outside of the tubular housing 330 and an inner cap portion 381, a distal portion of which is configured to fit within and engage the inside of the tubular housing 330. The inner and outer cap portions 381, 380 collectively serve to cap the open end of the tubular housing when the wand 310 is in the test position shown in FIGS. 13A and 13B. The cap housing 390 also includes an annular stem support collar 382 disposed within the inner cap portion 381. The stem support collar 382 surrounds and is fixedly attached to the proximal end 317 of the stem 310. The inner cap portion 381 and stem support collar 382 collectively define a second fluid reservoir 383 for retaining a second reagent liquid 352.

The handle 314 also includes a break-off nib 368 that extends proximally away from the proximal end 317 of the stem 310 through the second reservoir 383 into the first reservoir 372. The break-off nib 368 is connected to the proximal end of the inner cap housing 381 by a frangible closure 366 that serves to close off and prevent fluid communication between the first reservoir 372 and the second reservoir 383. The break-off nib 368 has a distal end 369 that is configured and positioned to block the opening into the stem tube flow passage 362 at the proximal stem end 317. This effectively prevents fluid from flowing into the flow passage 362.

The break-off nib 368 and the squeeze bulb 370 are configured so that when the squeeze bulb 370 and nib 368 are bent through at least a predetermined angle (typically 25 to 40 degrees) from the longitudinal axis 313 of the stem 210, the frangible closure 366 breaks away to allow fluid communication between the first and second reservoirs 372, 382. The break-off nib 368 is further configured so that once the closure 366 is broken, the nib is no longer constrained so as to prevent liquid from flowing into the passage 360. Once this is accomplished, the fluid passage 362 provides fluid communication between the first and second reservoirs 372, 383 and the test chamber 334. This provides a flow path past the seal 340 when the stem 310 is disposed there-through.

It will be understood that other selectively operable mechanisms may be provided to separate the first and second reservoirs 372, 383 until it is desirable to allow fluid communication between them. Likewise, any selectively operable mechanism may be used to close off the opening to the passage 362 until it is desirable to allow fluid communication between the reservoirs 372, 383 and the test chamber 334.

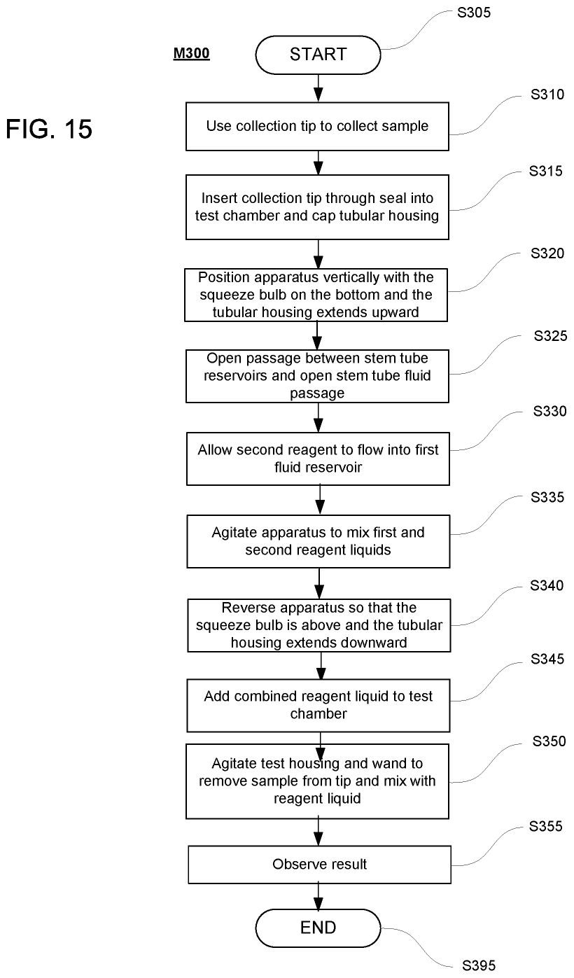

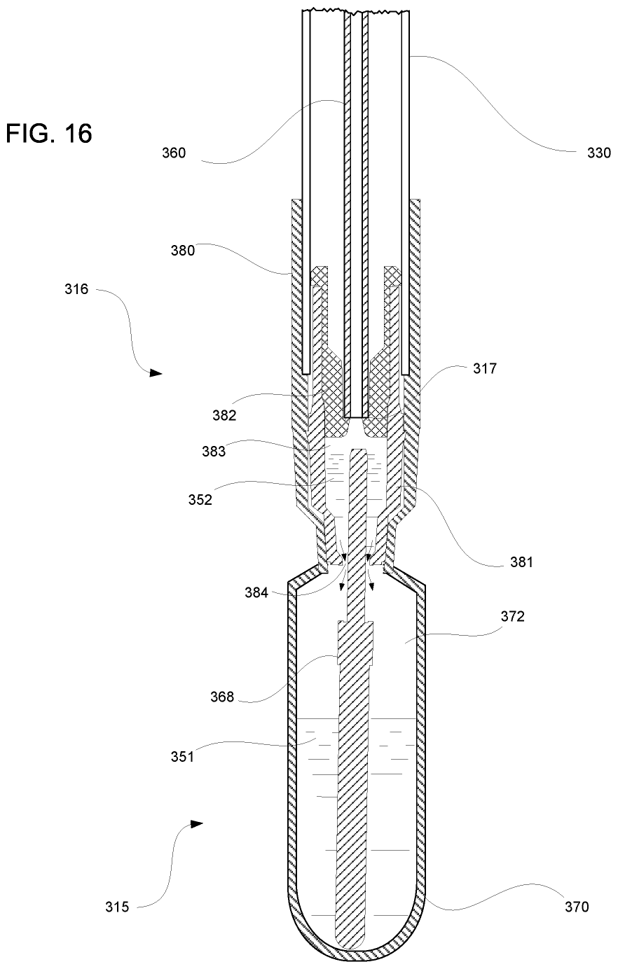

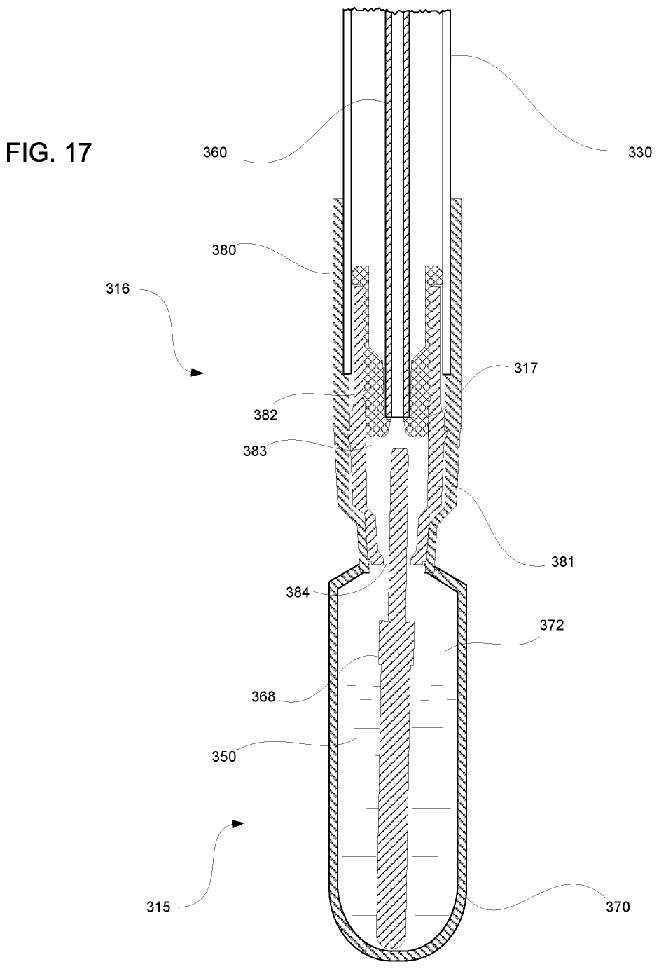

With reference to FIG. 15, a method M300 of using the collection and test apparatus 300 begins at S305. At S310, the user uses the collection tip 320 of the wand 310 to collect a sample of solid or semi-solid material. As before, this may typically be accomplished by rolling the collection tip 320 through the target material (e.g., stool) to adhere the material to the collection tip 320. The action may be repeated until all of the grooves of the tip 320 are filled with the target material. At S315, the user inserts the collection tip 320 through the open end of the test housing 330 and presses it through the aperture of the seal 340, thereby removing excess sample material from the collection tip 320. The user continues to move the tip distally into the test chamber 334 and into the test configuration, at which point the inner and outer cap housings 381, 380 engage and close off the proximal end of the tubular housing 330 as shown in FIGS. 13A and 13B. At S320, the user inverts the apparatus 300 so that the squeeze bulb 370 is on the bottom with the tubular housing 330 extending upward. At S325, the user opens the passage 384 between the first and second reservoirs 372, 383 by bending the squeeze bulb 370 and nib 368 to break the frangible closure 366. In some cases, this may require bending the nib 368 back and forth a number of times. With the closure 366 broken, the nib 368 is no longer held against the opening of the tube 360. The resulting configuration is shown in FIG. 16 in which flow from the second reservoir 383 into the first reservoir 372 is illustrated by arrows. By continuing to hold the apparatus 300 in the inverted configuration at S330, the user allows the second reagent liquid 352 to flow into the first reservoir 372 where it mixes with the first reagent liquid 351 to produce a combined reagent liquid 350, as shown in FIG. 17. At S335, the apparatus may be agitated by the user to fully mix the two reagent liquids 351, 352.

In some embodiments, there may be an observable change (e.g., in color) that allows the user to confirm that the two reagent liquids 351, 352 are fully mixed and the combined reagent liquid 350 is ready for testing. In such embodiments, it is advantageous that some or all of the squeeze bulb 370 be sufficiently transparent to allow observation of the change. In a particular embodiment, the first reagent liquid 351 is colorless when it is alone in the first reservoir 372. When the seal between the second reservoir 383 and the first reservoir 372 is broken, the combination of the first reagent liquid 351 and the second reagent liquid 352 produces a combined reagent liquid 350 that is a bright pink or fuchsia color that is readily observable within the bulb 370.

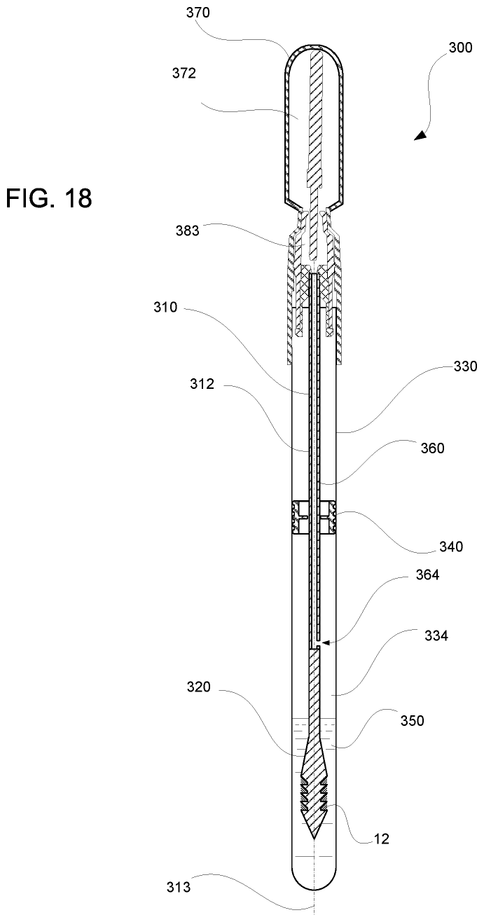

When the combined reagent liquid 350 is ready for application, the apparatus 300 is reversed at S340 so that the squeeze bulb 370 is above the tubular housing 330. At S345, the user adds the reagent liquid 350 to the test chamber 334 by compressing the squeeze bulb 370, thereby forcing the reagent liquid 350 out of the reservoir 372, into and through the passage 362 and out through the opening 364. Upon completion of this action, the reservoirs 372, 383 are drained and the sample-laden tip 320 is fully immersed in the reagent fluid 350 as shown in FIG. 18. At S350, the test housing 330 and wand 310 may be agitated to assure that all of the specimen material is removed from the collection tip 320 and mixed with the reagent liquid 350. At S355, the user observes or otherwise determines a test result based on the reaction of the specimen material with the reagent liquid 350. This could, for example, be a change in the color of the liquid 350, which would be observable through the wall of the tubular housing 330. Such a color change could be compared to a color key provided with the test apparatus 300. In particular embodiments, this color key could be applied to the housing 330 itself. The method ends at S395.

Many embodiments and adaptations of the present invention, other than those herein described with reference to the exemplary embodiments, will be apparent to those skilled in the art by the foregoing description, without departing from the substance or scope of the invention. While the present invention has been described herein in detail in relation to its exemplary embodiments, it is to be understood that this disclosure is only illustrative and exemplary of the present invention. Accordingly, the foregoing disclosure is not intended to be construed so as to limit the present invention or otherwise to exclude any other such embodiments, adaptations, variations, modifications, and equivalent arrangements. The claimed invention is limited only by the following claims.

What is claimed is:

1. A collection and test apparatus comprising:
a collection wand comprising
an elongate, cylindrical stem with a stem diameter, a stem axis, and proximal and distal stem ends, and
a collection tip extending distally from the distal stem end, the collection tip comprising a cylindrical sample capture portion having one or more specimen capture grooves formed therein and a capture portion diameter greater than or equal to the stem diameter, the one or more capture grooves each having a groove volume and collectively providing a specimen capture volume;
a tubular test housing having a housing interior, an open proximal end and a closed distal end, and a longitudinal housing axis extending through the proximal and distal ends; and
an annular seal member having a seal aperture sized to allow passage of the cylindrical sample capture portion of the collection tip there-through, the seal member being fixedly positioned within the tubular housing transverse to the longitudinal housing axis and dividing the housing interior into a proximal housing space and a distal housing space comprising a test chamber sized to receive at least a predetermined amount of reagent liquid
wherein the collection wand is positionable in a test configuration in which the stem axis is parallel to the longitudinal housing axis and the collection tip is disposed within the test chamber and the stem extends through the aperture seal and distally outward through the open proximal end of the test housing.

2. A collection and test apparatus according to claim 1 wherein the seal aperture has an undeformed seal diameter that is smaller than the capture portion diameter and the annular seal member is resiliently deformable material so that the seal aperture expands to allow passage of the cylindrical sample capture portion of the collection tip.

3. A collection and test apparatus according to claim 2 wherein
the capture portion diameter is greater than the stem diameter and
when the collection tip is received through the seal aperture, the seal aperture expands to the capture portion diameter to allow passage of the cylindrical sample capture portion then, after passage of the collection tip through the seal aperture, contracts to the greater of the undeformed seal diameter and the stem diameter.

4. A collection and test apparatus according to claim 1 wherein each specimen capture groove has a distal groove wall surface and a proximal groove wall surface and wherein at least one of the distal and proximal groove wall surface is oriented at an angle of less than 90 degrees relative to the stem axis.

5. A collection and test apparatus according to claim 4 wherein the distal groove wall surface is oriented at an angle of less than 90 degrees relative to the stem axis and the proximal groove wall surface is orthogonal to the stem axis.

6. A collection and test apparatus according to claim 1 wherein the collection tip is tapered from the cylindrical sample capture portion to a distal tip end.

7. A collection and test apparatus according to claim 1 wherein at least a portion of the stem is a tube having a stem fluid passage formed there-through, the stem fluid passage having a proximal stem passage opening through the proximal stem end and a distal passage opening formed through a wall of the tube at a location that is distal to the seal member when the collection wand is positioned in the test configuration.

8. A collection and test apparatus according to claim 7 wherein the collection wand comprises a handle portion attached at the distal end of the stem, the handle portion comprising a resiliently deformable squeeze bulb defining a reagent reservoir in fluid communication with the fluid passage.

9. A collection and test apparatus according to claim 7 wherein the collection wand comprises
a handle portion attached at the distal end of the stem, the handle portion comprising a resiliently deformable squeeze bulb defining a reagent reservoir;
a removable passage closure at the proximal stem passage opening, the passage closure preventing fluid flow through the proximal stem passage opening,
wherein removal of the passage closure places the test chamber in fluid communication with the reagent reservoir via the stem fluid passage.

10. A collection and test apparatus according to claim 9 wherein the removable passage closure comprises at least a portion of an elongate nib positioned so as to block the proximal stem passage opening, the elongate nib extending distally from the proximal stem passage opening so that at least a portion of the nib is disposed within the reagent reservoir.

11. A collection and test apparatus according to claim 10 wherein the elongate nib is positioned and configured so that bending deformation of the squeeze bulb and the elongate nib cause the elongate nib to separate from the proximal stem passage opening, thereby removing the passage closure.

12. A collection and test apparatus according to claim 7 wherein the collection wand comprises a handle having
a distal handle portion comprising a cap housing configured to engage and close the open proximal end of the tubular test housing when the collection wand is positioned in the test configuration, the cap housing surrounding the proximal stem end and defining a first reagent reservoir,
a proximal handle portion attached to and extending proximally from the distal handle portion and comprising a resiliently deformable squeeze bulb defining a second reagent reservoir connected to the first reagent reservoir by reservoir flow passage,
a frangible closure disposed intermediate the first and second reagent reservoirs and serving to prevent fluid flow through the reservoir flow passage, and
a removable passage closure at the proximal stem passage opening, the passage closure preventing fluid flow through the proximal stem passage opening,
wherein removal or breakage of the frangible closure places the first and second reagent fluid reservoirs in fluid communication via the reservoir flow passage and removal of the passage closure places the fluid passage in fluid communication with the reagent reservoir.

13. A collection and test apparatus according to claim 12 wherein
the removable passage closure comprises a first portion of an elongate nib positioned so as to block the proximal stem passage opening, the elongate nib extending distally from the proximal stem passage opening so that at least a proximal portion of the nib is disposed within the reagent reservoir,
the frangible closure comprises or is attached to a second portion of the elongate nib intermediate the first portion and the proximal portion.

14. A collection and test apparatus according to claim 13 wherein the elongate nib is positioned and configured so that bending deformation of the squeeze bulb and the elongate nib cause the frangible closure to break, thereby allowing fluid communication between the first and second reservoirs via the reservoir fluid passage, and cause the elongate nib to separate from the proximal stem passage opening, thereby removing the passage closure and allowing fluid communication between the first reagent reservoir and the test chamber via the stem flow passage.

15. A method of testing a solid or semi-solid target material using a collection and test apparatus having a collection wand and a tubular test housing with an annular seal disposed therein, the collection wand having a collection tip with a cylindrical sample capture portion with one or more specimen capture grooves collectively providing a specimen capture volume, and the tubular test housing being configured for receiving the collection tip through a proximal opening of the test housing and through a seal aperture of the annular seal member into a distal test chamber, the method comprising:
capturing target material by contacting the target material with the collection tip of the collection wand so as to at least completely fill each of the one or more specimen capture grooves;
placing a predetermined amount of reagent liquid in the distal test chamber;
inserting the collection tip through the proximal opening of the test housing;
passing the collection tip through the seal aperture, thereby removing captured target material adhered to the sample capture portion outside the specimen capture grooves;
positioning the collection wand in a test configuration in which the collection tip and remaining captured target material are disposed with the distal test chamber; and
dispersing the remaining captured target material in the reagent liquid.

16. A method according to claim 15 further comprising:
observing a result of a reaction between the captured target material and the reagent liquid.

17. A method according to claim 15 wherein the collection wand comprises a tubular stem having a proximal stem opening and a distal stem opening, the proximal stem opening being proximal to the proximal test housing opening and the distal stem opening being distal to the seal member when the collection wand is positioned in the test configuration, and wherein the action of placing a predetermined amount of reagent liquid is carried out after the action of positioning the collection wand.

18. A method according to claim 15 wherein the seal aperture has an undeformed seal diameter that is smaller than a diameter of the specimen capture portion and the annular seal member is resiliently deformable material so that the seal aperture expands to allow passage of the cylindrical sample capture portion of the collection tip.

19. A method of testing a solid or semi-solid target material using a collection and test apparatus having
a collection wand comprising a stem having stem fluid passage with a proximal stem passage opening and a distal stem passage opening, a collection tip having a cylindrical sample capture portion with one or more specimen capture grooves collectively providing a specimen capture volume, a handle including a deformable squeeze bulb defining a reagent reservoir having a reagent liquid disposed therein, and a removable passage closure closing the proximal stem passage opening and preventing fluid communication between the reagent reservoir and the stem fluid passage,
a tubular test housing having a housing interior, an open proximal end and a closed distal end; and
an annular seal member having a seal aperture, the seal member being fixedly positioned within the tubular housing and dividing the housing interior into a proximal housing space and a distal housing space comprising a test chamber,
wherein the collection wand is positionable in a test configuration in which the collection tip is disposed within the test chamber and the stem extends through the aperture seal and distally outward through the open proximal end of the test housing, and
wherein the distal passage opening is distal to the seal member when the collection wand is positioned in the test configuration so that removal or breakage of the removable closure allows fluid communication between the reagent reservoir and the test chamber,
the method comprising:
capturing target material by contacting the target material with the collection tip of the collection wand so as to at least completely fill each of the one or more specimen capture grooves;
inserting the collection tip through the proximal opening of the test housing;
passing the collection tip through the seal aperture, thereby removing captured target material adhered to the sample capture portion outside the specimen capture grooves;
positioning the collection wand in a test configuration in which the collection tip and remaining captured target material are disposed within the test chamber;
removing or breaking the removable closure to establish fluid communication between the reagent reservoir and the test chamber;
compressing the squeeze bulb to force reagent liquid through the stem flow passage into the test chamber to fully immerse the collection tip and remaining captured target material;
dispersing the remaining captured target material in the reagent liquid.

20. A method according to claim 19 further comprising:
observing a result of a reaction between the captured target material and the reagent liquid.

21. A method according to claim 19 wherein the seal aperture has an undeformed seal diameter that is smaller than a diameter of the specimen capture portion and the annular seal member is resiliently deformable material so that the seal aperture expands to allow passage of the cylindrical sample capture portion of the collection tip.

22. A method according to claim 19 wherein the removable passage closure comprises at least a portion of an elongate nib extending distally from the proximal stem passage opening so that at least a portion of the nib is disposed within the reagent reservoir, the elongate nib being positioned and configured so that bending deformation of the squeeze bulb and the elongate nib cause the removal or breakage of the passage closure and wherein the action of removing or breaking the removable closure includes:
bending the squeeze bulb and the elongate nib.

23. A method of testing a solid or semi-solid target material using a collection and test apparatus having
a collection wand comprising
a stem having stem fluid passage with a proximal stem passage opening and a distal stem passage opening,
a collection tip having a cylindrical sample capture portion with one or more specimen capture grooves collectively providing a specimen capture volume,
a handle including
a deformable squeeze bulb defining a first reagent reservoir having a first reagent liquid disposed therein
a cap housing defining a second reservoir having a second reagent liquid disposed therein, the second reservoir being distal to the first reservoir and connected to the first reservoir by a reservoir passage and having a passage connection to the proximal stem passage opening,
a first removable closure blocking the reservoir passage to prevent fluid communication between the first and second reservoirs,
a second removable closure blocking the proximal stem passage opening and preventing fluid communication between the second reagent reservoir and the stem fluid passage,
a tubular test housing having a housing interior, an open proximal end and a closed distal end, and
an annular seal member having a seal aperture, the seal member being fixedly positioned within the tubular housing and dividing the housing interior into a proximal housing space and a distal housing space comprising a test chamber,
wherein the collection wand is positionable in a test configuration in which the collection tip is disposed within the test chamber and the stem extends through the aperture seal and distally outward through the open proximal end of the test housing, and
wherein the distal passage opening is distal to the seal member when the collection wand is positioned in the test configuration so that removal or breakage of the removable closure allows fluid communication between the reagent reservoir and the test chamber,
the method comprising:
capturing target material by contacting the target material with the collection tip of the collection wand so as to at least completely fill each of the one or more specimen capture grooves;
inserting the collection tip through the proximal opening of the test housing;
passing the collection tip through the seal aperture, thereby removing captured target material adhered to the sample capture portion outside the specimen capture grooves;
positioning the collection wand in a test configuration in which the collection tip and remaining captured target material are disposed within the test chamber;
placing the collection and test apparatus in an orientation in which the second reagent reservoir is above the first reagent reservoir;
removing or breaking the first removable closure to allow the second reagent liquid to flow from the second reagent reservoir through the reservoir passage into the first reagent reservoir;
mixing the first and second reagent liquids in the first reagent reservoir to form a combined reagent liquid;
removing or breaking the second removable closure to establish fluid communication between the second reagent reservoir, the stem fluid passage and the test chamber;
inverting the collection and test apparatus so that the first and second reservoirs are above the test chamber;
compressing the squeeze bulb to force the combined reagent liquid out of the first reagent reservoir, through the second reservoir and the stem flow passage into the test chamber;
dispersing the remaining captured target material in the combined reagent liquid.

24. A method according to claim 23 further comprising:
observing a result of a reaction between the captured target material and the reagent liquid.

25. A method according to claim 23 wherein the seal aperture has an undeformed seal diameter that is smaller than a diameter of the specimen capture portion and the annular seal member is resiliently deformable material so that the seal aperture expands to allow passage of the cylindrical sample capture portion of the collection tip.

26. A method according to claim 23 wherein the actions of removing or breaking the first removable closure and removing or breaking the second removable closure are accomplished at the same time.

27. A method according to claim 26 wherein the second removable passage closure comprises at least a portion of an elongate nib extending distally from the proximal stem passage opening so that at least a portion of the nib is disposed within the first reagent reservoir, and wherein the elongate nib is attached to the first removable closure, the elongate nib being positioned and configured so that bending deformation of the squeeze bulb and the elongate nib causes the removal or breakage of both the first and second removable closures.
